(19) United States
(12) Patent Application Publication
(10) Pub. No.: US 2026/0102272 A1
(43) Pub. Date: Apr. 16, 2026

(54) POSTURE CORRECTOR AND GARMENT INCORPORATING THE SAME

(71) Applicant: Izabela Kogut, Rockaway Park, NY (US)

(72) Inventor: Izabela Kogut, Rockaway Park, NY (US)

(21) Appl. No.: 18/915,323

(22) Filed: Oct. 14, 2024

(51) Int. Cl. A61F 5/02; A41D 31/04

(52) U.S. Cl. CPC A61F 5/026; A41D 31/04

(57) ABSTRACT

A posture correcting mechanism including a first portion having a substantially trapezoidal configuration. A second portion having a substantially trapezoidal configuration, the second portion arranged directly longitudinally adjacent to and below the first portion. A third portion having an elongated, curved, rectangular configuration and comprising a first longitudinal end and a second longitudinal end, the third portion arranged directly laterally adjacent to the first and second portions at a first lateral side of the posture correcting mechanism. A fourth portion having an elongated, curved, rectangular configuration and comprising a first longitudinal end and a second longitudinal end, the fourth position arranged directly laterally adjacent to the first and second portions at a second lateral side of the posture correcting mechanism. A method of manufacturing the posture correction mechanism is also provided.

### TECHNICAL FIELD

This application is directed to posture correcting device.

### BACKGROUND

Known corrective apparel products are advertised as having the ability impact the wearer's physical appearance, for example, by placing the shoulders back in order to correct the wearer's posture. However, these products lack structural features that function to hold the shoulders back or provide concrete support.

If the shoulders are not properly aligned, the head will not be able to reposition, so the posture will not be changed or improved. If the shoulders are rounded, the pectoral muscles will remain tense and contracted (forward), which will position the head forward. Accordingly, there is a need for a device to correct bad posture from bent forward to neutral position.

### SUMMARY

An object of the present disclosure is to provide a garment that addresses the shoulder support that is a missing part in other correctors. Additionally, the other elements of the disclosure differ from the posture correctors that are currently available.

Another object of the present disclosure is to provide a posture corrector that applies forces to a wearer's body to properly align and balance the body to support and address body mechanics in order to achieve an improved posture.

Another object of the disclosure is to provide a posture corrector that is a wearable device utilizing a unique fabric design and construction that involves various portions of fabric that positioned along the back and shoulders to improve spinal biomechanics and scapular kinematics for better back support, better posture, increased chest expansion, and improved thoracic mobility. The technology of correcting element 10 provides a mechanism where each portion interacts or cooperates to produce a combined effect greater than the sum of the effect of each of the separate portions.

A posture correcting mechanism including a first portion having a substantially trapezoidal configuration. A second portion having a substantially trapezoidal configuration, the second portion arranged directly longitudinally adjacent to and below the first portion. A third portion having an elongated, curved, rectangular configuration and comprising a first longitudinal end and a second longitudinal end, the third portion arranged directly laterally adjacent to the first and second portions at a first lateral side of the posture correcting mechanism. A fourth portion having an elongated, curved, rectangular configuration and comprising a first longitudinal end and a second longitudinal end, the fourth position arranged directly laterally adjacent to the first and second portions at a second lateral side of the posture correcting mechanism.

A posture correcting mechanism including a first portion having a substantially trapezoidal configuration. A second portion having a substantially trapezoidal configuration, the second portion is arranged directly longitudinally adjacent to and below the first portion. A third portion has an elongated, curved, rectangular configuration and includes a first longitudinal end and a second longitudinal end, the third portion arranged directly laterally adjacent to the first and second portions at a first lateral side of the posture correcting mechanism. A fourth portion has an elongated, curved, rectangular configuration and includes a first longitudinal end and a second longitudinal end, the fourth portion arranged directly laterally adjacent to the first and second portions at a second lateral side of the posture correcting mechanism. A garment having a raglan sleeve configured for attachment with the third portion and the fourth portion.

A posture correcting mechanism including a first portion having a substantially trapezoidal configuration. A second portion having a substantially trapezoidal configuration, the second portion is arranged directly longitudinally adjacent to and below the first portion. A third portion has an elongated, curved, rectangular configuration and includes a first longitudinal end and a second longitudinal end, the first longitudinal end of the third portion is configured for positioning along the back of the shoulder blades and the second longitudinal end is configured for positioning on the front side below a collar bone, slightly above an armpit line, thereby crossing and pulling a shoulder joint, the third portion arranged directly laterally adjacent to the first and second portions at a first lateral side of the posture correcting mechanism. A fourth portion has an elongated, curved, rectangular configuration and includes a first longitudinal end and a second longitudinal end, the first longitudinal end of the fourth portion is configured for positioning along the back of the shoulder blades and the second longitudinal end is configured for positioning on the front side below a collar bone, slightly above an armpit line, thereby crossing and pulling a shoulder joint (glenohumeral joint), the first portion and the second portion are attached to third portion and fourth portion such that first and second portions are positioned along the shoulder blades (embody the medial borders of the scapulae and portions of the scapular fossae) of a user, the fourth portion arranged directly laterally adjacent to the first and second portions at a second lateral side of the posture correcting mechanism. A garment having a raglan sleeve configured for attachment with the third portion and the fourth portion.

### BRIEF DESCRIPTION OF THE DRAWINGS

The drawings presented herein show illustrative embodiments of the disclosure. They do not illustrate all embodiments. Other embodiments may be used in addition to or instead of the illustrative embodiments. Details that may be apparent or unnecessary may be omitted to save space or for more effective illustration. Some embodiments may be practiced with additional components or steps and/or without all the components or steps that are illustrated.

When the same numeral appears in different drawings, it refers to the same or like components or steps. The drawings are not intended to depict every feature of every implementation nor relative dimensions of the depicted elements and are not drawn to scale.

FIG. 1 is an illustration of a posture correcting device according to an exemplary embodiment of the present disclosure.

FIG. 2 is an illustration of a posture correcting device according to an embodiment of the present disclosure.

FIG. 3 is an illustration of portions of a posture correcting device according to an embodiment of the present disclosure.

FIG. 4 is an illustration of portions of a posture correcting device according to an embodiment of the present disclosure.

FIG. 5 is an illustration of a posture correcting device according to an embodiment of the present disclosure in use.

FIGS. 6a-6d is an illustration of a posture correcting device according to an embodiment of the present disclosure in use.

### DETAILED DESCRIPTION

In the following description, numerous specific details are set forth to clearly describe various exemplary embodiments disclosed herein. One skilled in the art, however, will understand that the subject matter of the present disclosure may be practiced without all of the specific details discussed below. In other instances, well-known features may not have been described so as not to obscure the disclosure with unnecessary detail regarding known features.

As used herein, the term “and/or” is to be interpreted broadly and is to be understood to refer to all or some of the elements. For example, “at least one of (a) and/or (b)” means the teaching pertains only to element (a), or only to element (b), or to both element (a) and element (b). In another example, “at least one of (a), (b), and/or (c)”, means the teaching pertains to only element (a), or only to element (b), or only element (c), or to elements (a) and (b), elements (a) and (c), elements (b) and (c), or to all of (a), (b) and (c).

Exemplary embodiments of the present disclosure are directed to a posture correcting mechanism and a garment that incorporates the mechanism. The technology of the posture correction garment addresses bad habits, muscle memory (interaction between the brain and the nervous system), and proprioception (based on sensory receptors in the skin, joints, and muscles that signal changes in stretch to the nervous system) related and work together to develop and establish an even distribution and balance of muscle tone in a biomechanical way without creating an over tense struggle, thereby eliminating a sensation of being put in a straight jacket. The technology activates the body's system with the interference in the correlation and interconnection of the head, neck, shoulders and the entire back.

This corrective device offers an instant recognition of an incorrect posture and an automatic stimulation towards transformation, redistribution of the muscle effort throughout the body, executing improved musculoskeletal alignment and body mechanics. The posture correction garment will facilitate a hindrance and termination of a bad habit/posture.

The technology works by pulling the shoulders back and opening the pectoral muscles, thereby letting the head move back. This activates the scapulas (shoulder blades) to contract, resulting in stronger upper back muscles. This will strengthen the muscles between shoulder blades: the rhomboid major and rhomboid minor to maintain a good posture.

The garment creates a sensation of wearing a brace, however, body movements and flexibility are not restricted. The corrector does not serve as an immobilizer; it is not too tight or rigid (allows full range of movement). In contrast, it encourages the muscles to strengthen without weakening or wasting the muscles or causing other biomechanical problems. The technology of the posture correction garment addresses bad habits and muscle memory to develop and establish an even distribution and balance of muscle tone in a biomechanical way without creating an over tense struggle. Thereby eliminating a sensation of being put in a straight jacket. The technology activates the body's system with the interference in the correlation and interconnection of the head, neck, shoulders and the entire back.

The front elements work synergistically with the back elements by pulling the shoulders back and bringing the left and right scapula together, narrowing the distance between them. The back (posterior part) of the mechanism is addressing scapular retraction, this movement involves pulling the shoulder blades towards each other and towards the spine, and doesn't shrug the shoulders upwards. The construction and the specific placement of the bands in the corrective mechanism create sophisticated tension to facilitate biomechanically correct stance.

The garment allows subtle movements and contractions of the upper body muscles to strengthen them in a very gentle continuous manner until an improvement is established and a positive change in body mechanics is achieved.

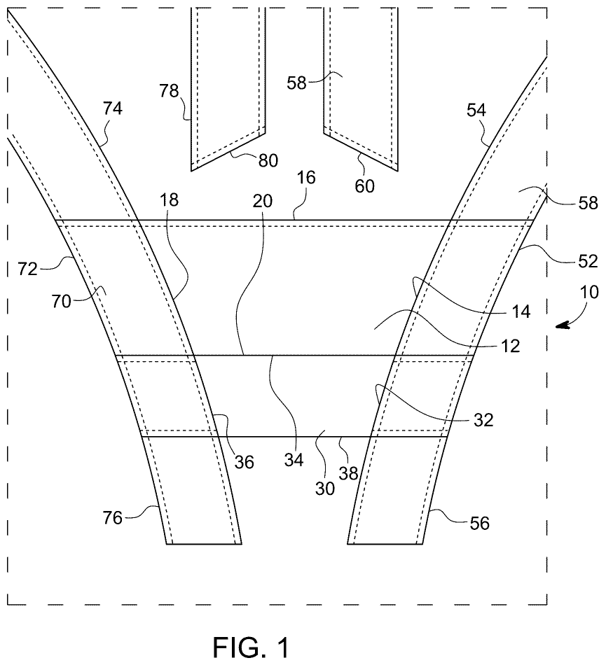

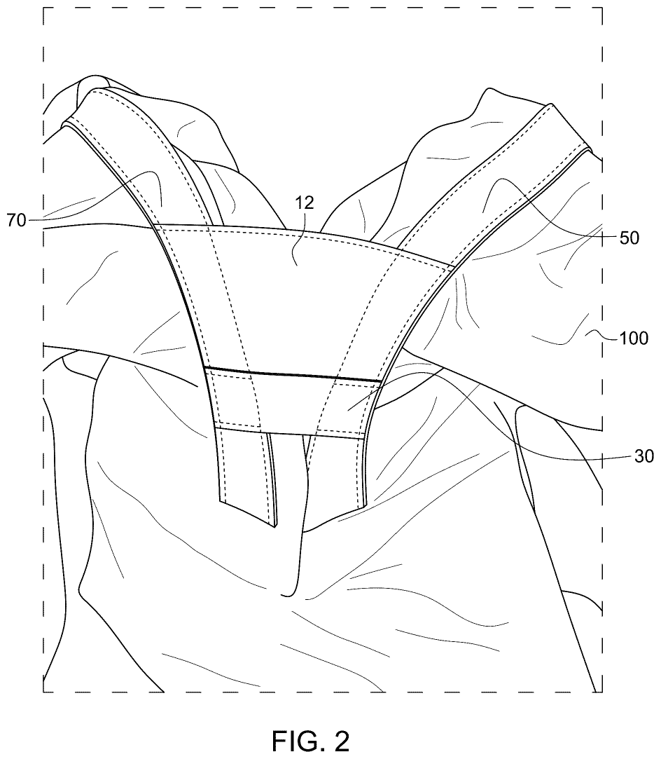

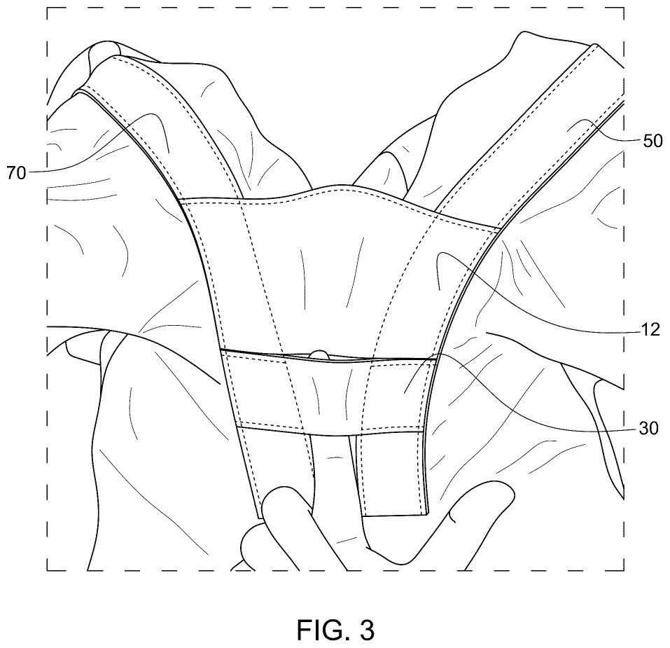

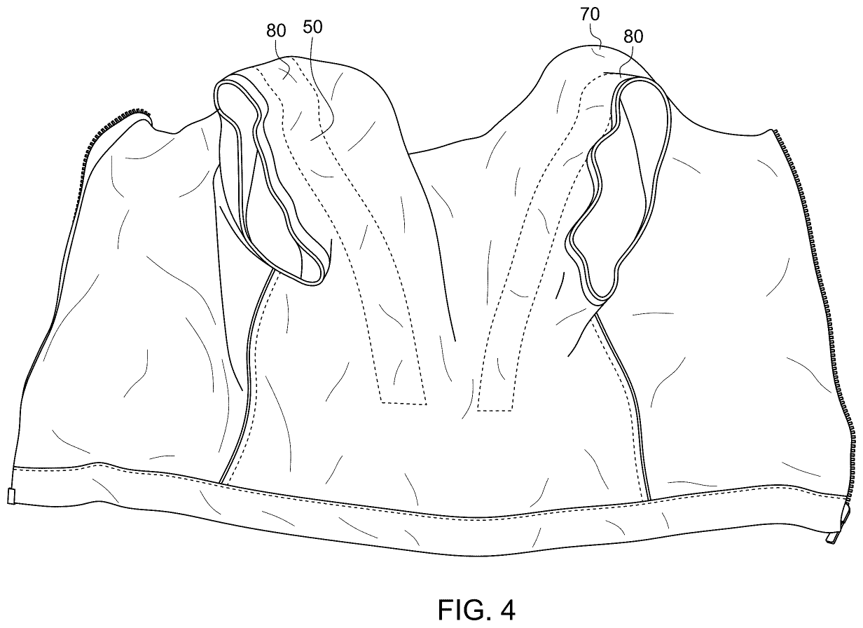

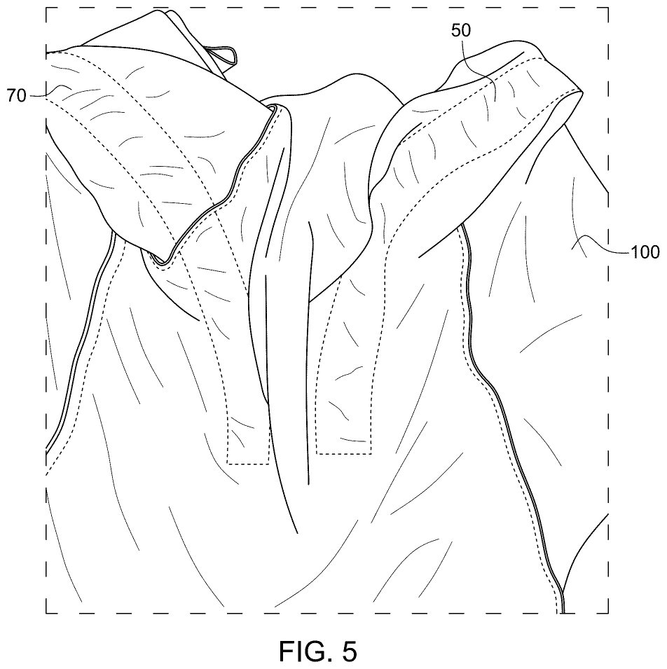

FIG. 1 shows a posture correcting mechanism, generally designated by reference number 10, according to an exemplary embodiment of the present disclosure. The posture correcting mechanism 10 includes a first portion 12, a second portion 30, a third portion 50 and a fourth portion 70. First portion 12 has a generally trapezoidal shape (inverted trapezoid), and includes a first side 14, a second side 16, a third side 18 and a fourth side 20. Second portion 30 also has a generally trapezoidal shape (inverted trapezoid), and includes a first side 32, a second side 34, a third side 36 and a fourth side 38. The second portion 30 is arranged directly longitudinally adjacent to and below the first portion 12.

Third portion 50 has an elongated, curved, rectangular configuration, and includes a first side 52 and a second side 54. The third portion 50 is arranged directly laterally adjacent to the first side 14 of the first portion 12 and the first side 32 of the second portion 30, and curves outwardly relative to the longitudinal center line of the mechanism 10. The third portion 50 extends longitudinally above and below the first and second portions 12, 30.

Third portion 50 includes a first longitudinal end 56 and a second longitudinal end 58. First longitudinal end 56 is configured for positioning along the back between the shoulder blades. Second longitudinal end 58 is configured for positioning on the front side below the collar bone, slightly above the armpit line, thereby crossing and pulling the shoulder joint. Second longitudinal end 58 has an angled edge 60 so that the edge slopes downwards away from the centerline of the mechanism 10.

Fourth portion 70 has the same general configuration as the third portion 50, with the exception that it is arranged directly laterally adjacent to the third side 18 of the first portion 12 and the third side 36 of the second portion 30. In this regard, the fourth portion 70 is arranged laterally opposite from the third portion 50 and curves outwardly relative to the longitudinal centerline of the mechanism 10 in a direction opposite to the direction in which the third portion 50 is curved. The fourth portion 70 includes a first side 72, a second side 74, a first longitudinal end 76 and a second longitudinal end 78.

The first longitudinal end 76 is configured for positioning along the back between the shoulder blades. The second longitudinal end 78 is configured for positioning on the front side below the collar bone, slightly above the armpit line, thereby crossing and pulling the shoulder joint. The second longitudinal end 78 has an angled edge 80 so that the edge slopes downwards away from the centerline of the mechanism 10.

First portion 12 and second portion 30 are attached to third portion 50 and fourth portion 70 such that first and second portions 12, 30 are positioned along the shoulder blades medial borders of the scapulae of the user. The first side 14 of the first portion 12 is attached to the second side 54 of the third portion 50. The third side 18 of the first portion 12 is attached to the second side 74 of the fourth portion 70. The first side 32 of the second portion 30 is attached to the second side 54 of the third portion 50. The third side 36 of the second portion 30 is attached to the second side 74 of the fourth portion 70. The attachments may be formed by, for example, sewing, gluing, and/or fastening.

Third and fourth portions 50 and 70 are made of a material configured to pull the shoulder blades together. The third and fourth portions 50 and 70 are configured to pull the shoulders back to facilitate the movement and action of first and second portions 12 and 30. The entire length of third and fourth portions 50 and 70 is adhered to the garment. Only the ends of portions 12 and 30 are adhered to portions 50. 70 and integrated with portions 50. 70 to the garment. Approximately 20% to 30% of portion 30 is not adhered to the garment. Approximately 40% to 50% of portion 12 is not adhered to the garment. These percentages of portions 12, 30 not being adhered to the garment allows portions 12, 30 to apply a synergistic force to the scapula to draw the shoulder blades together. The placement of portions 12, 30 has been extensively tested and tried on users and shown to apply a greater horizontal force to the scapulas to facilitate pulling the shoulder blades together.

In exemplary embodiments, the mechanism 10 is configured to be integrated into a garment 100, such as, for example, a garment that has a raglan sleeve. The raglan style is important for third portion 50 and fourth portion 70 because the raglan sleeve is created with a continuous piece of fabric, extending from the collar of the garment to the underarm. This provides a diagonal seam that is suitable for attachment with angled edge 60 and angled edge 80. Edges 60, 80 merge at the front with the raglan sleeve, for example, edge 60 and edge 80 are cut at an angle of approximately 30 degrees. They are incorporated into the garment, and they initiate the pull of the shoulders. The portions of the correcting element 10 are made of a solid strong fabric, such as, for example, nylon, spandex, polyester, power mesh, elastane, neoprene rubber, no-roll woven elastic, knitted elastic, heavy weight & wide elastic band, Lycra etc. Correcting element 10 supports and complements the entire corrective action. The portions are enclosed by a non-toxic material incorporated into a garment. In addition, the material contains anti-microbial moisture wicking and protects against ultra-violet rays. Additionally, the fabric has a specific weight 200-250 GSM (GSM stands for grams per square meter, used to measure the weight of fabric. Fabrics with a higher GSM are generally thicker and more durable), it plays an important role in supporting/complementing the whole system. The mechanism is aesthetically appealing and is applicable for use for females and males incorporated into an adequate wear style. All elements of the corrective technology can be of various lengths and widths depending upon the size of the wearer. The spectrum of wearers varies from busy professionals, individuals who suffer from work related risk prevention to teenagers (being self-conscious and struggling with body image issues) and the aging cohort (preventing posture deterioration due to physical decline). This can be worn as an inner layer or as an outer garment. The user will slightly notice the working mechanism when the muscle memory of a correct posture is attained. It will in an accepting manner/passively encourage an active motion of scapular retraction along with shoulders and more to positively affect the user's body mechanics. The anterior and posterior elements (support for both front and back) provide the strongest, well adjusted amount of tension to achieve a desired outcome—correct posture. The non-toxic fabric for example, offered by Pine Crest Fabric (e.g. FlexSoft) enclosing the mechanism, received the OEKO-Tex Standard 100 Certification, certifying no harmful chemicals are present in the material of the fabric (OEKO-Tex STANDARD 100 is a label for textiles tested for harmful substances). The intention of the mechanism besides serving its purpose is additionally holistic. Chemicals used in textiles can enter the body (absorption through the skin), they can cause a variety of health issues, including Endocrine system interference, Cancer, Nervous System damage, the liver, and more. This approach has never been adopted in previous products/posture correctors. Additionally, the unique invention design comprises elastic/pre-tensioned bands/straps that encourages posterior pull (originated anteriorly) and horizontal posterior stretch/contraction to facilitate proprioceptive postural alignment—musculoskeletal balance of the neck, shoulders, scapulas, and the back. Both parts: anterior and posterior work synergistically and collectively.

The present correcting element 10 is a wearable device utilizing a unique fabric design and construction that involves various portions of fabric that positioned along the back and shoulders to improve spinal biomechanics and scapular kinematics for better back support, better posture, increased chest expansion, and improved thoracic mobility. The technology of correcting element 10 provides a mechanism where each portion interacts or cooperates to produce a combined effect greater than the sum of the effect of each of the separate portions.

The technology of correcting element 10 corrects rounded shoulders, forward head posture (FHP) and prevents temporomandibular joint disorders (TMJ), thoracic outlet syndrome (TOS). Poor posture causes nerve compression which leads to TOS (if resulting from bad posture, there are other factors that can cause TOS (physical trauma, tumors, anatomical defects and more).

Upper crossed syndrome (UCS) is often caused by poor posture, such as sitting or standing with the head positioned forward for a prolonged time. UCS causes a muscular imbalance in the upper body that can lead to pain, stiffness and reduced range of motion. The syndrome is caused by overuse of the muscles around the neck, shoulders and upper back. Some symptoms of USC include Tightness or pain in the neck, shoulders and upper body. Headaches, restricted range of motion in the neck and shoulders. Numbness, tingling and pain in the upper arms. Difficulty sitting, reading, watching TV, or prolonged driving. In UCS, weak become cervical flexors and rhomboid muscles along with the middle and lower trapezius muscles. Tight become suboccipital (group of four neck muscles that help extend and rotate head, and support the head and neck's posture), upper trapezius and levator scapula muscles along with pectorals muscles.

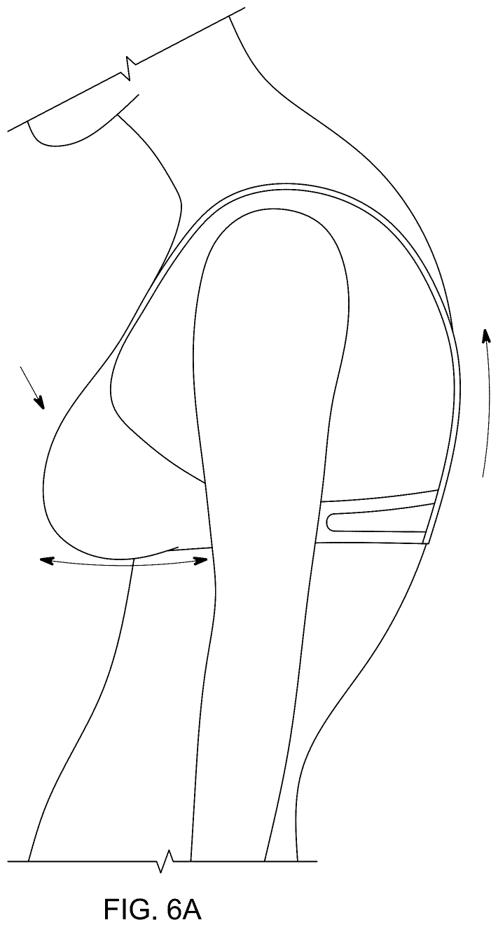

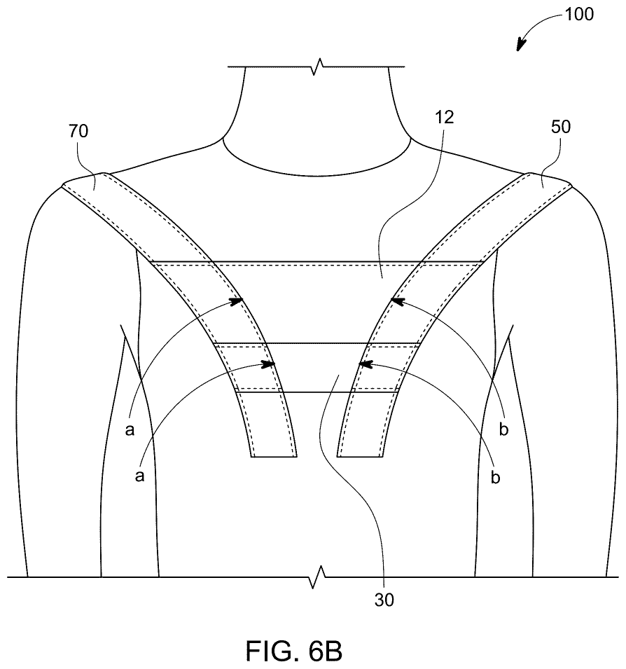

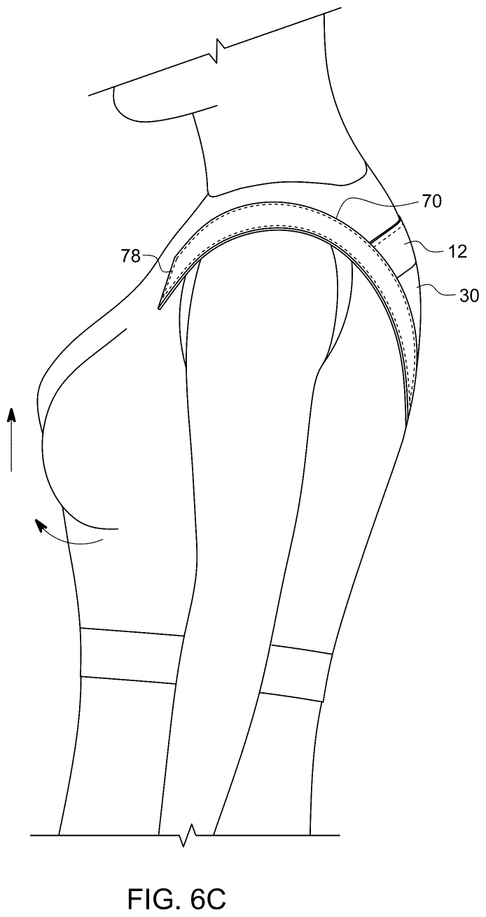

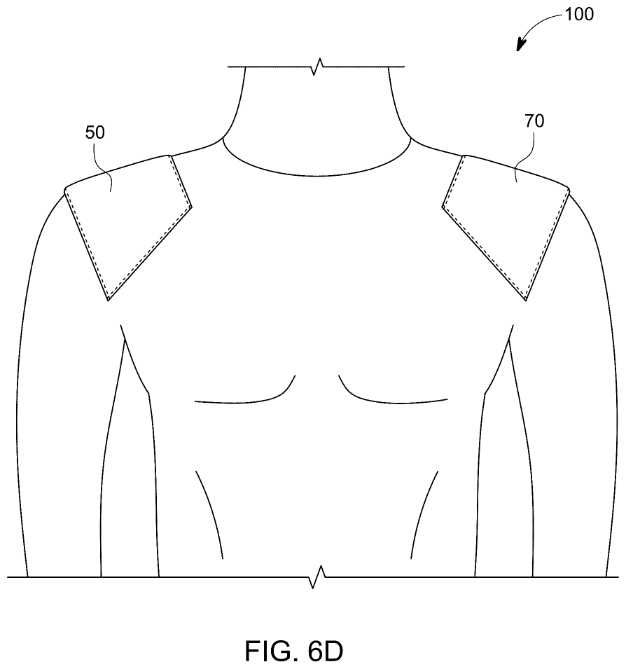

FIGS. 6a-6d show the correcting element 10 in use. FIG. 6a simulates a hunched over kyphotic posture: noticeable alteration of scapular & shoulder position, the chest is compromised, and the upper abdominal muscles shorten (protruding stomach). FIG. 6a shows a typical round shoulder posture that certain people succumb to either with age, trauma or other medical issues. The shoulders round forward and the head juts forward, which causes neck, spine, and shoulder pain. Correcting element 10 is positioned in the two areas needed to correct bad posture, along the shoulder blades, as shown in FIG. 6b and in front of the shoulders, as shown in FIG. 6c. Portion 12 and portion 30 contract, as shown by arrows A and B in FIG. 6b, to pull the shoulder blades together to open the deltoids (anterior) and the pectoral muscles. They work synergistically and collectively with the front parts to facilitate this action. They re-align the scapulas from a protracted (forward) position to a retracted (horizontal) position. A horizontal movement (horizontal tension) of the elements 12 and 30 (FIG. 6b) moves the scapulas towards the spine prompting the spinal extension and arranging a space for the head resulting in its realignment on top of the spine. This facilitated musculoskeletal action activates three groups of muscles: the iliocostalis, longissimus, and spinalis (known as the spinal erectors); crucial in supporting and maintaining posture by steadying the spine on the pelvis during walking. The shoulders remain down and away from the ears due to the induced relaxation of the trapezius. The interplay between shoulder, scapular and spinal biomechanics regains a user's correct posture and keeps thriving. FIG. 6c simulates an anatomically correct posture: the kyphosis doesn't prevail, the lumbar region is straight, chest expanded, shoulder in neutral position along with the head, and the abdominal area is flat. The breathing comes easier, the tension on the neck is reduced along with the shoulders, decreased back pain and improved overall spinal condition.

Portion 50 and portion 70 each apply a force to the front of shoulders via end 58 and end 78 (end 78 not seen in FIG. 6c), as shown by arrow C in FIG. 6c, to pull shoulders back to further contract the shoulder blades and open the deltoids (anterior) and the pectoral muscles. As portion 12, portion 30, portion 50 and portion 70 apply forces to the shoulder blades and the front of the shoulders the potions work in conjunction to achieve a neutral head position and facilitate pulling the shoulders back, and retracting/bringing scapulae together.

The corrective element 10 also addresses other issues, for example, it can relieve neck, shoulder and back pain, improve body mechanics, balance issues, increases oxygen intake (chest expansion), impacts facial bones and muscles/overall appearance of the face. The corrective element 10 may also provide psychological benefits, for example, improve self-esteem and body image, a dignified/self-confident manner of walking. Occupation risk prevention (various industries & healthcare), and athletic & sports: optimizing human performance in space. Improving and preventing progressive slouching by using the corrective mechanism can have other positive effects on the body, including: pain reduction (bad posture can apply extra pressure on the spinal discs, which leads to pain in the neck, shoulders, and back); improved digestion (slouching may put pressure on the abdominal area, causing heartburn, and slow digestion); improved lung capacity (bad posture can limit lung capacity). In addition, poor posture compromises skeletal muscle range of motion and strength which can predispose an individual to repetitive injuries. The corrective mechanism can enhance spinal and shoulder proprioception, scapular and glenohumeral kinematics, improve thoracic mobility and facilitate improved breathing. Also, improved posture will enhance bone density and body composition, by distributing/rebalancing weight evenly throughout the body, which can reduce risk of osteoporosis. Additionally, correct posture decreases strain on the muscles, tissues, and bones in the back, hips, and knees. Consequently, this can prevent wear and tear which can create a joint inflammation or damage in the longer run. Also, good posture helps muscles function properly, warranting that the muscles along with the tendons and ligaments work efficiently to provide proper support and stabilization for the bones. Poor posture can also negatively impact the lymphatic system: reduced lymphatic drainage. Slouching, forward head posture or upper crossed syndrome (discussed above) can reduce lymphatic drainage in the neck and will cause more strain on the posterior neck muscles (suboccipital muscles, splenius capitis, and semispinalis capitis). When lymphatic drainage in the neck is compromised, it could result in lymphedema (chronic swelling that results from a buildup of fluid in the tissues). All well-known lymphatic drainage techniques, and even the natural lymphatic drainage process, will not be fully successful unless the posture is aligned. The corrective mechanism 10, addresses a proper head alignment, this will allow the lymph fluid to perfuse smoothly, preventing blockages. Correctly aligned posture is the foundation of the body considering: health benefits, efficient movement, positive self-image, and well-being.

While several illustrative embodiments of the present disclosure have been shown and described, numerous variations and alternative embodiments will occur to those skilled in the art. Such variations and alternative embodiments are contemplated and can be made without departing from the scope of the disclosure as defined in the appended claims.

As used in this specification and the appended claims, the singular forms “a,’ ‘an,” and “the” include plural referents unless the content clearly dictates otherwise. The term plurality” includes two or more referents unless the content clearly dictates otherwise. Unless defined otherwise, all technical and scientific terms used herein have the same meaning as commonly understood by one of ordinary skill in the art to which the disclosure pertains.

The foregoing detailed description of exemplary and preferred embodiments is presented for purposes of illustration and disclosure in accordance with the requirements of the law. It is not intended to be exhaustive nor to limit the disclosure to the precise form(s) described, but only to enable others skilled in the art to understand how the disclosure may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the disclosure be defined by the Claims as written and equivalents as applicable Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the claims.

What is claimed is:

1. A posture correcting mechanism comprising:
a first portion having a substantially trapezoidal configuration;
a second portion having a substantially trapezoidal configuration, the second portion arranged directly longitudinally adjacent to and below the first portion;
a third portion having an elongated, curved, rectangular configuration and comprising a first longitudinal end and a second longitudinal end, the third portion arranged directly laterally adjacent to the first and second portions at a first lateral side of the posture correcting mechanism; and
a fourth portion having an elongated, curved, rectangular configuration and comprising a first longitudinal end and a second longitudinal end, the fourth position arranged directly laterally adjacent to the first and second portions at a second lateral side of the posture correcting mechanism.

2. The posture correcting mechanism of claim 1, wherein the third portion extends longitudinally above and below the first and second portions.

3. The posture correcting mechanism of claim 1, wherein the fourth portion extends longitudinally above and below the first and second portions.

4. The posture correcting mechanism of claim 1, wherein the first portion includes a first side, a second side, a third side and a fourth side.

5. The posture correcting mechanism of claim 4, wherein the second portion includes a first side, a second side, a third side and a fourth side.

6. The posture correcting mechanism of claim 5, wherein the third portion is positioned directly laterally adjacent to the first side of the first portion and the first side of the second portion.

7. The posture correcting mechanism of claim 5, wherein the fourth portion is positioned directly laterally adjacent to the third side of the first portion and the third side of the second portion.

8. The posture correcting mechanism of claim 1, wherein the first portion and the second portion are attached to the third portion and the fourth portion such that first and second portions are positioned along the shoulder blades of a wearer of the posture correcting mechanism.

9. The posture correcting mechanism of claim 4, wherein the first side of the first portion is attached to the second side of the third portion, the third side of the first portion is attached to the second side of the fourth portion, the first side of the second portion is attached to the second side of the third portion, the third side of the second portion is attached to the second side of the fourth portion.

10. The posture correcting mechanism of claim 1, wherein the first, second, third and fourth portions are enclosed by a non-toxic material incorporated into a garment.

11. The posture correcting mechanism of claim 1, wherein, when the posture correcting
mechanism is worn by a wearer, the first longitudinal end of the third portion is positioned at the wearer's posterior side along shoulder blades of the wearer and the second longitudinal end is positioned at the wearer's anterior side below a collar bone, and above an armpit line, thereby crossing and pulling a shoulder joint (glenohumeral joint) of the wearer in a posterior direction.

12. The posture correcting mechanism of claim 1, wherein, when the posture correcting mechanism is worn by a wearer, the first longitudinal end of the fourth portion is positioned at the wearer's posterior side along shoulder blades of the wearer, and the second longitudinal end is positioned at the wearer's anterior side below a collar bone, and above an armpit line, thereby crossing and pulling a shoulder joint (glenohumeral joint) of the wearer in a posterior direction.

13. The posture correcting mechanism of claim 1, wherein the second longitudinal end of the third portion includes an angled edge (configured to have an edge of ≈30°) that slopes downwards away from a centerline of the mechanism.

14. The posture correcting mechanism of claim 13, wherein the second longitudinal end of the fourth portion includes an angled edge of approximately 30 degrees that slopes downwards away from a centerline of the mechanism.

15. The posture correcting mechanism of claim 14, further comprising a garment attachment.

16. The posture correcting mechanism of claim 15, wherein the garment includes a raglan sleeve having a diagonal seam configured for attachment with the angled edges of the third portion and the fourth portion.

17. A posture correcting mechanism comprising:
a first portion having a substantially trapezoidal configuration;
a second portion having a substantially trapezoidal configuration, the second portion is arranged directly longitudinally adjacent to and below the first portion;
a third portion has an elongated, curved, rectangular configuration and includes a first longitudinal end and a second longitudinal end, the third portion arranged directly laterally adjacent to the first and second portions at a first lateral side of the posture correcting mechanism;
a fourth portion has an elongated, curved, rectangular configuration and includes a first longitudinal end and a second longitudinal end, the fourth portion arranged directly laterally adjacent to the first and second portions at a second lateral side of the posture correcting mechanism; and
a garment having a raglan sleeve configured for attachment with the one or more portions.

18. A posture correcting mechanism comprising:
a first portion having a substantially trapezoidal configuration;
a second portion having a substantially trapezoidal configuration, the second portion is arranged directly longitudinally adjacent to and below the first portion;
a third portion has an elongated, curved, rectangular configuration and includes a first longitudinal end and a second longitudinal end, the first longitudinal end of the third portion is configured for positioning along the back along the shoulder blades and the second longitudinal end is configured for positioning on the front side below a collar bone, slightly above an armpit line, thereby crossing and pulling a shoulder joint, the third portion arranged directly laterally adjacent to the first and second portions at a first lateral side of the posture correcting mechanism;
a fourth portion has an elongated, curved, rectangular configuration and includes a first longitudinal end and a second longitudinal end, the first longitudinal end of the fourth portion is configured for positioning along the back to facilitate contracting of the shoulder blades and the second longitudinal end is configured for positioning on the front side below a collar bone, slightly above an armpit line, thereby crossing and pulling a shoulder joint, the first portion and the second portion are attached to third portion and fourth portion such that first and second portions are positioned along the back of the shoulder blades of a user, the fourth portion arranged directly laterally adjacent to the first and second portions at a second lateral side of the posture correcting mechanism; and
a garment having a raglan sleeve configured for attachment with one or more portions.
